(19) United States
(12) Patent Application Publication
(10) Pub. No.: US 2015/0157310 A1
(43) Pub. Date: Jun. 11, 2015

(54) ROTARY MEDICAL ANCHOR, AND MEDICAL KIT COMPRISING SAID ANCHOR

(71) Applicants: Jean-Yves Paul Albert Coillard-Lavirotte, Saint Cyr au Mont D'or (FR); Daniel Edmond Boublil, Champagne au Mont D'or (FR); Philippe Emmanuel D'Ingrado, Saint Didier au Mont D'or (FR)

(72) Inventors: Jean-Yves Paul Albert Coillard-Lavirotte, Saint Cyr au Mont D'or (FR); Daniel Edmond Boublil, Champagne au Mont D'or (FR); Philippe Emmanuel D'Ingrado, Saint Didier au Mont D'or (FR)

(21) Appl. No.: 14/405,095

(22) PCT Filed: Jun. 5, 2013
(86) PCT No.: PCT/FR2013/051272

§ 371 Date: Dec. 2, 2014

(30) Foreign Application Priority Data
Jun. 5, 2012 (FR) 1255231

(51) Int. Cl. A61B 17/04

(52) U.S. Cl. CPC A61B 17/0401; A61B 2017/0409; A61B 2017/0414

(57) ABSTRACT

The invention relates to a medical anchor (1) for implanting in a bone mass by impacting, said anchor comprising a main body (2) of longitudinal axis X-X′ with firstly a distal portion (3) for penetration and secondly a proximal portion (4) for acting as an interface with an installation instrument (5), said anchor also including anchor means (6) and being characterized in that the anchor means (6) are stationary, projecting radially from the main body (2), and are shaped to provide the anchor (1) with anchoring in the bone mass on said anchor being turned, the main body (2) being cylindrical in shape with a distal portion (3) that is terminated by a penetration cone (16) with a pointed end. Surgical device.

### TECHNICAL FIELD

The present invention relates to the general technical field of medical devices for use in the field of surgery by surgeons when performing surgery for the purpose of fastening or repairing biological tissues such as ligaments relative to biological supports such as bones.

More precisely, the present invention relates to the field of surgery and in particular orthopedic surgery and/or reconstructive surgery.

The present invention relates to a medical anchor for implanting in a bone mass by impacting, said anchor comprising a main body of longitudinal axis X-X′ with firstly a distal portion for penetration and secondly a proximal portion for acting as an interface with an installation instrument, said anchor also including anchor means.

### PRIOR ART

Medical anchors are already well known, and they are medical devices that are used, e.g. in orthopedic surgery, to enable a surgeon to have connection points for implanting a suture by means of which the surgeon can then fasten biological tissues, in particular ligaments.

Medical anchors are thus particularly useful for enabling a surgeon to fasten or refasten one or more ligaments, or more generally one or more biological tissues after traumatic accidents such as sprains or lax joints.

Medical anchors are also particularly useful for changing the insertion position of one or more ligaments in the context of performing surgery.

At present there exist several types of anchor device suitable for being used by surgeons.

Firstly there are conventional anchors for screwing that are in the general form of a screw that is provided, e.g. towards its proximal end, with an eye through which a suture can be passed and thus serve as attachment means.

In that type of anchor, it is the screw thread that bites into and bears against the bone mass, thereby preventing the device from being extracted and producing the looked-for anchoring effect.

Such anchor devices generally give satisfaction, but in certain situations, depending on the trauma, it is found that it is not appropriate, and often not even possible, to make use of such anchors that are implanted by screw means. By way of example, there may be a danger of cracking or micro-cracking, which may take place immediately, or in the short term after the anchor has been installed, for example.

Anchoring screws of that type also present other drawbacks. Thus, installing such screws also makes use of an operating technique that is relatively lengthy and that depends on the number of screw threads.

Furthermore, most surgery involving the installation of a surgical screw is performed by mini-invasive techniques so that the surgeon does not have the possibility of seeing the implant. There thus exists a non-negligible risk of the anchor not being properly installed and of it being engaged within the bone mass either too much or too little.

Anchor devices are also known that operate by elastic deformation, which devices are installed by being impacted into the bone mass and returned to their initial shape after the impacting stage, such that the anchor is securely anchored in the bone mass against a withdrawal movement in the direction opposite to the impacting direction.

So-called “umbrella” anchors are anchors of this type, and they are constituted by numerous small rods that are folded during the stage of penetration into the bone, and that then spread out like umbrella ribs so as to become blocked against the cortical bone and prevent the anchor being extracted when a traction force is exerted on the sutures.

Those anchor devices generally give satisfaction, but they are not suitable for being used in all situations and for all traumas that might be suffered, particularly since such devices may require recourse to a series of several installation instruments in succession, thereby complicating the work of the surgeon.

Umbrella anchors also suffer from the drawback of being prevented from opening correctly or completely when they are put into place in bone masses that are particularly hard and that are stronger than the deployment force required for proper and complete opening of the umbrella anchor. Naturally, that is very harmful to proper retention of the anchor.

Finally, anchor devices are known that require an appropriate housing to be made initially in the bone with the help of suitable tool, followed by the anchor being inserted in the housing, and finally by the anchor being tilted in order to obtain its final anchoring.

Although that type of device gives satisfaction under certain surgical conditions, it is found not to be usable under all surgical situations that are encountered, and it involves a sequence of operations and tools that make it complicated to install anchors of that type.

It is also found that although a surgeon has good control over the stage of inserting tilting anchors, the subsequent tilting stage is not controllable by a surgeon since it depends solely on the ratio between the force delivered by the instrument that performs the tilting and the quality of the bone mass. During the terminal stage of withdrawing the instrument, tilting can thus fail to occur, such that the anchor can be extracted freely from its housing without the surgeon being able to take action to prevent such extraction.

Consequently, the objects given to the invention seek to provide a remedy to the various above-mentioned drawbacks and to propose a novel medical anchor that, while being very versatile in use and adaptable to a wide variety of surgical situations, is particularly simple to use and provides an anchoring effect that is effective.

Another object of the invention seeks to propose a novel medical anchor that is particularly robust and simple to use.

Another object of the invention seeks to provide a novel medical anchor that is particularly easy to implant.

Another object of the invention seeks to propose a novel medical anchor that can be installed reliably and without risk of its position becoming degraded over time.

Another object of the invention seeks to provide a novel medical anchor that can be installed quickly.

Finally, the invention seeks to propose a novel medical kit enabling the medical anchor to be implanted quickly and effectively.

### SUMMARY OF THE INVENTION

The objects given to the invention are achieved with the help of a medical anchor for implanting in a bone mass by impacting, said anchor comprising a main body of longitudinal axis X-X′ with firstly a distal portion for penetration and secondly a proximal portion for acting as an interface with an installation instrument, said anchor also including anchor means and being characterized in that the anchor means are stationary, projecting radially from the main body, and are shaped to provide the anchor with anchoring in the bone mass on said anchor being turned, the main body being cylindrical in shape with a distal portion that is terminated by a penetration cone with a pointed end.

The objects given to the invention are achieved with the help of a medical kit constituted by a medical anchor in accordance with the invention and an instrument for installing said medical anchor in the bone mass of a patient.

### BRIEF DESCRIPTION OF THE DRAWINGS

Other features and advantages of the invention appear and can be seen in greater detail on reading the following description made with reference to the accompanying drawings that are given purely by way of non-limiting illustrative example, and in which:

FIG. 1 is a general side view of an assembly in accordance with the invention comprising a medical anchor in accordance with the invention together with an installation instrument, together forming a medical kit in accordance with the invention;

FIG. 2 is a plan view showing a medical anchor in accordance with the invention;

FIG. 3 is a fragmentary side view in perspective of an embodiment detail of the invention, showing a medical anchor in accordance with the invention co-operating with the end of an installation instrument in accordance with the invention;

FIG. 4 is a perspective view showing a medical anchor in accordance with the invention; and

FIG. 5 is a side view showing a medical anchor in accordance with the invention.

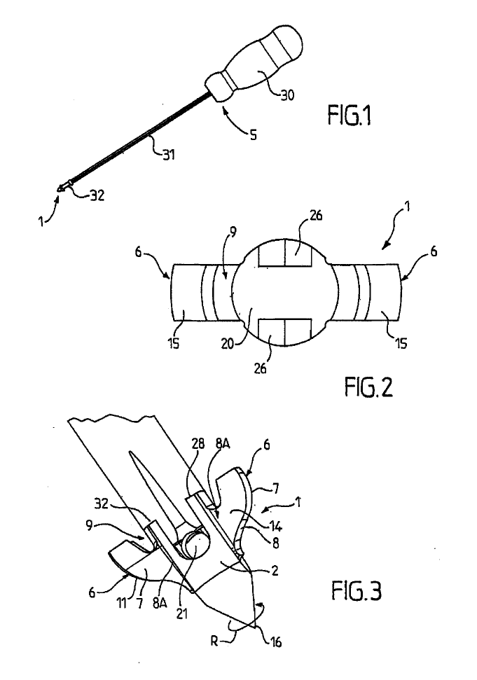

### BEST MANNER OF PERFORMING THE INVENTION

As shown in the figures, the medical anchor 1 in accordance with the invention is for implanting on, within, or under a bone mass (not shown) by being impacted, i.e. the surgeon must begin by exerting an axial compression force on the medical anchor 1 that is sufficient to enable the anchor to penetrate by force into the bone mass so that the anchor can penetrate fully into the bone mass over a depth greater than the overall height of said medical anchor 1.

For this purpose, the medical anchor 1 has a main body 2, e.g. in the form of a cylinder defining a longitudinal axis X-X′, having a distal portion 3 for penetration and a proximal portion 4 for acting as an interface with an instrument 5 for installing said medical anchor 1.

As shown in the figures, the medical anchor 1 of the invention also has anchor means 6 that are stationary, projecting radially from the main body 2 and shaped to anchor the anchor 1 in the bone mass when said anchor 1 is turned.

Thus, in the invention, the anchor means 6 occupy a defined or final position that is stationary relative to the main body 2, i.e. the anchor means are not movable and not retractable, and thus form an integral portion of the main body 2.

The anchor means 6 also extend radially outwards from the main body 2 so as to constitute external elements extending in a direction different from the longitudinal axis X-X′ so as to form engagement elements for anchoring the medical anchor 1 in the bone mass when said anchor is turned about the longitudinal axis X-X′ after being impacted, i.e. once the medical anchor 1 has been able to penetrate into and within the bone mass.

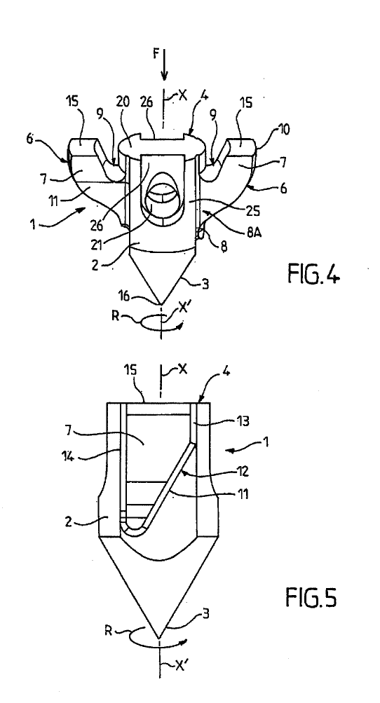

These structural characteristics thus enable the medical anchor 1 to be blocked in position, and after being turned against movement in a direction opposite from the impacting direction (reference F in FIG. 4), such that after they have been turned, the anchor means 6 can become blocked under the cortical bone without returning along the path created by the anchor means 6 during the action of being impacted through the bone mass.

In a particularly preferred version of the invention, the anchor means 6 are formed by fins 7 as shown in the figures.

In the invention, the fins 7 may have various geometrical configurations without thereby going beyond the ambit of the invention, it being understood that they need to have a general shape and a profile that facilitates penetration into the bone mass during impacting, and that also facilitates turning so as to end up ensuring good retention within the bone mass.

For this purpose, and as shown in the figures, the fins 7 are connected to the periphery of the main body 2 via a connection zone 8A, and they may present a connection profile 8 in the thickness of the fin 7 that possesses marked progressiveness such that the fins 7 move progressively in regular or other manner away from the periphery of the main body on going from the distal portion 3 and the connection zone 8A towards the proximal portion 4.

In a preferred version of the invention, as shown in the figures, the medical anchor of the invention has two fins 7 extending symmetrically in diametrically opposite directions relative to the longitudinal axis X-X′ of the main body 2, i.e. on either side thereof.

As shown in the figures, the two fins 7 are identical and they are preferably separate from the main body 2 in their proximal zones 10 so as to form two channel portions or slots 9 that are arranged between the periphery of the main body and the proximal portions 10. The fins 7 are thus secured to the main body 2 only via their connection zones 8A so that they present a certain amount of springiness, mainly in their proximal zones 10 level with the slots 9, thus facilitating penetration during the impacting stage.

Nevertheless, in a variant, fins 7 in accordance with the invention need not be separate from the main body 2 in their proximal zones 10, so as to form fins 7 of the non-separated delta type, by analogy with delta wings as used in aviation.

In a particularly advantageous version of the invention, the fins 7 present respective sloping flats 11 that enhance impacting and turning of the medical anchor 1 about the axis X-X′.

As shown, in particular in FIG. 5, the sloping flats 11 are situated on the distal zones 12 of the fins 7, the sloping flats 11 of each of the fins 7 being situated on the same side relative to the thickness of each fin 7, which thickness defines a front face 13 and a rear face 14 of each fin 7. Since the sloping flat 11 forms the terminal portion of the front face 13 in the distal zone 12 of each fin 7, the two sloping flats 11 serve not only to provide the surgeon with an easily identifiable visual reference for determining the conventional direction of rotation (referenced R) of the medical anchor 1, but also provide mechanical assistance during the impacting stage and while turning the medical anchor 1, making it easier to turn.

According to another advantageous characteristic of the invention, the anchor means 6 extend radially from the main body 2 and are terminated towards the proximal portion 4 by plane faces 15 that extend in planes that are orthogonal to the axis X-X′.

Because of this feature, the medical anchor 1 of the invention has a large surface area in the zone coming into contact with the cortical bone, thereby serving to reduce the risk of perforation when a traction force is applied, and thus presenting maximum strength.

In particularly advantageous manner, and as shown, in particular in FIGS. 4 and 5, the plane faces 15 extend at the same level as the proximal portion 4 of the main body 2.

As shown, the main body is generally in the form of a substantially regular cylindrical body with a distal portion 3 that is terminated by a penetration cone 16 having a preferably pointed end, while the opposite portion of the cylindrical body is formed by the proximal portion 4 that is in the form of a plane surface 20 that is preferably coplanar with the plane faces 15.

The penetration cone 16 is pointed in shape, its tip preferably presenting an acute angle so that said penetration cone 16 is sharp so as to enable it to penetrate into the patient's bone while the medical anchor 1 is being impacted therein. When an axial force and/or at least one axial impact is exerted on the medical anchor 1 towards the bone that is to be impacted, said medical anchor 1 can itself penetrate through the cortical wall of the bone. In preferred manner, the fins 7 are also sharp so that they too are capable of penetrating into the patient's bone when an axial force and/or at least one axial impact is exerted on the medical anchor 1.

The medical anchor 1 of the invention is thus self-perforating.

According to other characteristics of the invention, the medical anchor 1 has an orifice 21 for passing a suture (not shown), said orifice 21 extending through the main body 2 in a direction orthogonal to the axis X-X′.

The orifice 21 is preferably centered in the anchor and presents openings that are machined so as to avoid the suture being cut in the event of being put under tension.

Thus, in the invention, the medical anchor comprises:
a main body 2 of cylindrical shape having a distal portion 3 that is terminated by a penetration cone 16;
a penetration cone 16 that is surmounted by a middle portion 25 through which a through orifice 21 is formed, the anchor means 6, 7 projecting radially from said middle portion 25; and
a middle portion 25 that is surmounted by a proximal portion 4 that lies at the same level as the plane faces 15 of the anchor means 6, 7.

In particularly advantageous manner, the proximal portion 4 is provided with notches 26 in the form of longitudinal setbacks made parallel with the axis X-X′ in the mass of the cylindrical body 2 starting from the proximal portion 4 and extending as far as the orifice 21 and its open flanks.

The notches 26 are for co-operating with corresponding notch-engaging means 28 (FIG. 3) of the instrument 5 for installing the medical anchor for the purposes of providing impacting and turning of the medical anchor 1.

The installation instrument 5, similar to a screwdriver type gripper hand tool, is provided with a handle 30 from which there extends a substantially cylindrical rod 31 having its end 32 provided with the notch-engaging means 28 of shape and dimensions enabling them to be inserted axially in the notches 26 with sufficient retention friction to enable the surgeon to take hold of the medical anchor 1 and clamp it to the installation instrument 5 in order to perform impacting followed by turning of the medical anchor 1 in accordance with the invention.

Thus, the invention also provides a medical kit made up of a medical anchor 1 in accordance with the invention and an installation instrument 5 for installing said medical anchor in the bone mass of a patient.

The medical kit in accordance with the invention comprises the instrument 5 formed by a gripper provided at one end with notch-engaging means 28 for co-operating with notches, specifically the notches 26 of the medical anchor 1 in accordance with the invention in order to hold it prior to putting it into place by impacting followed by turning in the bone mass.

The medical anchor in accordance with the invention operates as follows.

After ensuring that the medical anchor 1 is gripped by the instrument 5 by inserting the notch-engaging means 28 axially into the notches 26, the surgeon can cause the medical anchor 1 to penetrate into the bone mass by impacting. For this purpose, the surgeon causes the medical anchor 1 to penetrate axially along the axis X-X′ by exerting an axial force F such that the medical anchor 1 can penetrate into the bone mass to a sufficient depth for its distal penetration portion 3 to ensure that the entire anchor 1 penetrates into the bone mass.

Once penetration has been performed by impacting, the surgeon turns the anchor about the axis X-X′ by turning the instrument 5 in the appropriate direction R for turning the medical anchor 1 as indicated by the presence of the sloping flats 11. The medical anchor 1 is preferably turned through a single quarter turn, it nevertheless being understood that it is possible to use an angle that is significantly smaller since it suffices merely to reach an angular position such that the fins 7, and in particular their plane faces 15, come into contact with a retention bone mass enabling the device to be anchored.

Thereafter, the surgeon can separate the instrument 5 from the medical anchor 1 and withdraw the instrument.

The invention also provides a method of installing a medical anchor in the bone mass of a patient, the method comprising the following steps:
causing the anchor to penetrate into and under the cortical bone mass along an impacting axis during an impacting step; and
then turning the anchor about the impacting axis X-X′ in order to block the anchor under the cortical bone mass.

In particularly advantageous manner, the anchor is caused to penetrate into and under the bone mass to a depth that is greater than the overall height of the anchor.

In the invention, the anchor is then turned through an angle of value smaller than 180°, and preferably of the order of 90° starting from the position it occupies as the result of impacting.

In the invention, it is also possible to facilitate the impacting step by beginning by making a housing in the bone mass. Thus, in the invention, prior to the step of impacting the anchor, a housing is made in the bone mass in order to serve as a guide for the anchor during impacting.

This housing is made by a surgeon using any appropriate means, for example with the help of a drill bit of appropriate diameter for making a cylindrical housing of depth greater than the overall height of the anchor.

A housing is then made of diameter that is less than the overall diameter of the anchor so as to leave in place a portion of bone mass suitable for anchoring the fins 7.

In the method of the invention, the steps of impacting and of turning the anchor are both performed using the same gripper tool 5 after securing the anchor to the gripper tool.

In the invention, the medical anchor 1 may be put into place with a suture previously engaged through the orifice 21, it naturally being equally possible for the suture to be the resorbable type or of the non-resorbable type.

Finally, the medical anchor of the invention is made out of any material that is suitable for medical use, and for example it may be made out of a metal alloy such as titanium, stainless steel, or cobalt-chromium, or made out of thermoplastics materials such as polyetheretherketone (PEEK).

Medical anchors of the invention may be made with various sizes, e.g. lying in the range 2.0 millimeters (mm) in diameter to 5.0 mm in diameter.

### SUSCEPTIBILITY OF INDUSTRIAL APPLICATION

Industrial application of the invention lies in the field of medical anchors for being put into by impacting in order to fasten or repair biological tissues relative to biological supports.

1. A medical anchor (1) for implanting in a bone mass by impacting, said anchor comprising a main body (2) of longitudinal axis X-X′ with firstly a distal portion (3) for penetration and secondly a proximal portion (4) for acting as an interface with an installation instrument (5), said anchor also including anchor element (6) and being characterized in that the anchor element (6) is stationary, projecting radially from the main body (2), and is shaped to provide the anchor (1) with anchoring in the bone mass on said anchor being turned, the main body (2) being cylindrical in shape with a distal portion (3) that is terminated by a penetration cone (16) with a pointed end.

2. A medical anchor (1) according to claim 1, characterized in that the anchor element (6) is formed by fins (7).

3. A medical anchor (1) according to claim 2, characterized in that each fin (7) presents a sloping flat (11) enhancing impacting and turning of the anchor (1).

4. A medical anchor (1) according to claim 2, characterized in that it has two fins (7) extending in diametrically opposite directions relative to the longitudinal axis X-X′ of the main body (2).

5. A medical anchor (1) according to claim 1, characterized in that the two fins (7) are identical and separate from the periphery of the main body (2) in their proximal zones (10) so as to form two channel portions or slots (9) formed between the periphery of the main body (2) and the proximal portions (10).

6. A medical anchor (1) according to claim 1, characterized in that the anchor element (6) extends radially from the main body (2) and is terminated towards the proximal portion (4) by plane faces (15) that extend in planes orthogonal to the axis (X-X′).

7. A medical anchor (1) according to claim 1, characterized in that it is provided with a through orifice (21) for a suture, said orifice (21) extending through the main body (2) in a direction orthogonal to the axis (X-X′).

8. A medical anchor (1) according to claim 1, characterized in that the proximal portion (4) is provided with notches (26) for co-operating with corresponding notch-engaging element (28) of an instrument (5) for installing the medical anchor by impacting and turning the anchor.

9. A medical anchor (1) according to claim 6, characterized in that:
the penetration cone (16) is surmounted by a middle portion (25) through which the through orifice (21) is formed, the anchor element (6) projecting radially from said middle portion (25); and
the middle portion (25) is surmounted by the proximal portion (4), which extends at the same level as the plane faces (15) of the anchor means (6).

10. A medical kit constituted by a medical anchor (1) according to claim 1 and an instrument (5) for installing said medical anchor (1) in the bone mass of a patient.

11. A medical kit according to claim 10, characterized in that the instrument (5) is formed by a gripper having notch-engaging element (28) at one end for co-operating with notches (26) of the medical anchor (1) in order to retain the medical anchor prior to installing it by impacting it and then turning in the bone mass.

12. A medical anchor (1) according to claim 3, characterized in that it has two fins (7) extending in diametrically opposite directions relative to the longitudinal axis X-X′ of the main body (2).

13. A medical anchor (1) according to claim 2, characterized in that the two fins (7) are identical and separate from the periphery of the main body (2) in their proximal zones (10) so as to form two channel portions or slots (9) formed between the periphery of the main body (2) and the proximal portions (10).

14. A medical anchor (1) according to claim 3, characterized in that the two fins (7) are identical and separate from the periphery of the main body (2) in their proximal zones (10) so as to form two channel portions or slots (9) formed between the periphery of the main body (2) and the proximal portions (10).

15. A medical anchor (1) according to claim 4, characterized in that the two fins (7) are identical and separate from the periphery of the main body (2) in their proximal zones (10) so as to form two channel portions or slots (9) formed between the periphery of the main body (2) and the proximal portions (10).

16. A medical anchor (1) according to claim 2, characterized in that the anchor element (6) extends radially from the main body (2) and is terminated towards the proximal portion (4) by plane faces (15) that extend in planes orthogonal to the axis (X-X′).

17. A medical anchor (1) according to claim 3, characterized in that the anchor element (6) extends radially from the main body (2) and is terminated towards the proximal portion (4) by plane faces (15) that extend in planes orthogonal to the axis (X-X′).

18. A medical anchor (1) according to claim 7, characterized in that:
the penetration cone (16) is surmounted by a middle portion (25) through which the through orifice (21) is formed, the anchor element (6) projecting radially from said middle portion (25); and
the middle portion (25) is surmounted by the proximal portion (4), which extends at the same level as the plane faces (15) of the anchor element (6).

19. A medical kit constituted by a medical anchor (1) according to claim 9 and an instrument (5) for installing said medical anchor (1) in the bone mass of a patient.

20. A medical kit according to claim 19, characterized in that the instrument (5) is formed by a gripper having notch-engaging element (28) at one end for co-operating with notches (26) of the medical anchor (1) in order to retain the medical anchor prior to installing it by impacting it and then turning in the bone mass.
